(19) United States
(12) Patent Application Publication
(10) Pub. No.: US 2021/0177266 A1
(43) Pub. Date: Jun. 17, 2021

(54) INTRAORAL SCANNING WITH RAW DEPTH DATA

(71) Applicant: Clayton Adams Teufel, Chicago, IL (US)

(72) Inventor: Clayton Adams Teufel, Chicago, IL (US)

(21) Appl. No.: 17/125,673

(22) Filed: Dec. 17, 2020

Related U.S. Application Data
(60) Provisional application No. 62/949,112, filed on Dec. 17, 2019.

(51) Int. Cl. A61B 5/00; A61B 90/00; B33Y 80/00; B33Y 10/00

(52) U.S. Cl. CPC A61B 5/0088; A61B 5/0086; A61B 5/002; A61B 2090/062; A61B 90/06; B33Y 80/00; B33Y 10/00; A61B 5/682

(57) ABSTRACT

The following relates generally to production of dental products. More particularly, techniques are disclosed for generating a 3D model of a patient's mouth, which may be used to produce dental products. In some embodiments, an infrared scanner is used to generate the raw depth information of a patients mouth. The produced dental product may include any of dental dentures, implants, teeth, aligners, crowns, veneers, partials, relines, mouthguards, retainers, etc.

### CROSS REFERENCE TO RELATED APPLICATIONS

This application claims the benefit of U.S. Provisional Patent Application No. 62/949,112, entitled “Intraoral Scanning with Raw Depth Data,” filed Dec. 17, 2019, which is expressly incorporated by reference herein in its entirety.

### BACKGROUND

Remote dentistry (or “tele-dentistry”) is in growing demand as technology is allowing cost-effective remote access to healthcare providers. Many patients do not have the financial ability to receive dental care, many do not desire to go (or fear going) to a dentist, Covid is causing trepidation in receiving in-office dental care, and/or many do not have convenient access to a dental professional. Tele-dentistry can mitigate all of these issues.

Additionally, technology is allowing direct-to-consumer delivery of dental-related products and services. The technology described herein further allows consumers to perform at-home scans of their teeth and mouth which may be used to provide dental products and services.

Intraoral scanners are an integral means to capture patients' dental data (digital dental impressions) which can be transmitted via the Internet for use in tele-dentistry. Historically the high cost of intraoral scanners limited the use of such scanners to the professional dental community. The technology described herein reduces the costs of intraoral scanning to a point which makes it accessible on a direct to consumer basis.

For example, increasingly, people are seeking to correct mild malocclusion with clear dental aligners that can be provided by an orthodontist, a dentist, in a retail location or even direct to consumer via the Internet or other sales marketing channels (e.g., television advertising). It is estimated that over sixty percent (60%) of the U.S. population does not have convenient access to an orthodontist for traditional malocclusion treatments (e.g., “braces”). Additionally, many people cannot afford such treatments and can benefit from both the convenience and cost savings of such clear aligner treatments.

In addition to increasing demand to correct mild malocclusion, it is estimated that over 178 million Americans are missing one or more teeth and require dentures (including partial dentures) or dental implants to replace missing teeth. Yet, only one million or so Americans receive dentures or implants each year. Many of those with missing teeth lack access to dental services due primarily to prohibitively high costs, lack of dental offices within commuting distance and/or lack of access to oral scanners to capture dental data. An impediment to providing greater dental care, and most notably tele-dentistry, is the high cost and lack of availability of intraoral scanners to capture digital dental data. It is estimated that as few as 15% of dentists have intraoral scanners, and the remainder use difficult-to-administer impression kits. Oral scanning platforms offered by incumbent commercial companies typically cost between $15,000 and $30,000. They use expensive, high-resolution, high-frequency RGB cameras that require upwards of 500 images “stitched” together to create a scan. Post-processing requires expensive, high-end hardware components and the results are still often unusable by treatment designers. Many of these manufacturers also provide lists of approved desktop and laptop hardware that add significantly to the cost of the platform while restricting use of the data. They also often provide strict licensing requirements for the included software. A lower cost scanner may also be sold direct-to-consumer, rather than through expensive, multi-layered distribution channels.

The systems and methods disclosed herein provide solutions to these problems and others.

### SUMMARY

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

In one aspect, there is an intraoral scanning system for generating depth data to be used to produce a dental product (including, but not limited to a clear aligner, a partial denture, a full denture, a dental implant, a tooth, a surgical guide or tray or any other dental product). The system may include: a scanner communicatively coupled with one or more processors, the scanner comprising: (i) an emitter configured to output an emission, and (ii) a receiver configured to receive the emission and generate a scanning signal based on the received emission; the one or more processors a may be configured to generate the depth data based on the scanning signal.

In the system as described in the preceding paragraph, the scanner may be an infrared scanner, the emitter may be an infrared emitter (which emitter may include an infrared “projector” capable of emitting numerous “dots” versus one single data point or “dot”), and the receiver may be an infrared receiver that can capture the data (or “dots”) sent from the emitter. The scanner may be a laser scanner, the emitter may be a laser emitter, and the receiver may be a laser receiver. The scanner may also be an Red, Green, Blue (RGB) sensor capable of capturing color data. The emitter may be a first emitter, and the receiver may be a first receiver; and the scanner may further include: (i) a second emitter configured to output a second emission, and (ii) a second receiver configured to receive the second emission and generate a second scanning signal based on the second emission. The scanner may be communicatively coupled to the one or more processors via either a wired or wireless communication, including but not limited to wi-fi or Bluetooth. The system may further include a mouthguard including the scanner; and the emitter may be a first emitter, and the receiver may be a first receiver; and the scanner may further comprise: (i) a second emitter configured to output a second emission, and (ii) a second receiver configured to receive the second emission and generate a second scanning signal based on the second emission.

In another aspect, there is a method for producing a dental product, the method comprising: using a scanner to scan a mouth of a patient, the scanner comprising (i) an emitter or projector configured to output an emission, and (ii) a receiver configured to receive the emission and generate a scanning signal based on the received emission; sending the generated scanning signal to one or more scanning processors; with the one or more scanning processors, generating depth data of the patient's mouth; and sending the depth data from the one or more scanning processors to one or more dental production processors.

The method as described in the preceding paragraph may further include producing the dental product according to the depth data. The method may further include producing the dental product according to the depth data, and not according to any image data from a camera. The scanner may be an infrared scanner, the emitter may be an infrared emitter or infrared projector, and the receiver may be an infrared receiver. The scanner may be a laser scanner, the emitter may be a laser emitter, and the receiver may be a laser receiver. The emitter may be a first emitter, and the receiver may be a first receiver; and the scanner may further include (i) a second emitter configured to output a second emission, and (ii) a second receiver configured to receive the second emission and generate a second scanning signal based on the second emission; and the one or more scanning processors may generate the depth data further based on the second scanning signal. The generated scanning signal may be sent to the one or more scanning processors via wired or wireless communication, including but not limited to wi-fi or Bluetooth. The method may further include producing the dental product: according to the depth data; and using a 3D printer. The method may further include producing the dental product according to the depth data; and the produced dental product may include one of a dental denture, implant, aligner, crown, veneer, partial, reline, mouthguard, or retainer.

In yet another aspect, there is an intraoral scanner for producing depth data of a patient's mouth, the intraoral scanner comprising: an infrared emitter or infrared projector configured to output an infrared emission; and an infrared receiver configured to receive the infrared emission and generate a scanning signal based on the received infrared emission.

The systems and methods disclosed herein advantageously have a lower financial cost than prior systems.

A further advantage of the systems and methods disclosed herein is that less processing is required (e.g., prior RGB camera systems required upwards of 500 images to be processed).

### BRIEF DESCRIPTION OF THE DRAWINGS

FIG. 1A shows an example of an intraoral scanning system including one emitter and one receiver.

FIG. 1B shows an example of an intraoral scanning system with a scanner shaped as a toothbrush.

FIG. 2 shows an example of an intraoral scanning system including more than one emitter and receiver.

FIG. 3 shows an example of an intraoral scanning system using wireless communication.

FIG. 4 shows an embodiment involving a mouthguard.

FIG. 5 shows an example scan produced by an app in accordance with the systems and methods described herein.

FIG. 6 shows an example of an intraoral scanning system sending information to a dental manufacturing facility.

FIG. 7 shows an example embodiment of a handle including a mirror and an IR projector and receiver.

FIG. 8 shows an example embodiment of a handle including a mirror and an RGB camera.

FIG. 9A shows an example of a scanner with a headpiece.

FIG. 9B shows an example of a scanner without a headpiece.

FIG. 10 shows two examples of 3D printed dentures.

FIG. 11 illustrates an additional bottom and side view of the scanner.

FIG. 12 illustrates an additional top and side view of the scanner.

FIG. 13 illustrates and additional bottom view of the scanner.

FIG. 14 illustrates an additional top view of the scanner.

FIG. 15 illustrates an additional side view of the scanner.

FIG. 16 illustrates an additional side view of the scanner.

FIG. 17 illustrates an additional front view of the scanner.

FIG. 18 illustrates an additional rear view of the scanner.

### DETAILED DESCRIPTION

The present embodiments relate to, inter alia, producing dental products using depth information. For instance, depth information of a patient's mouth (e.g., including depth information of the patient's teeth, gums, arches, one structure and so forth) may be acquired using an infrared scanner. The depth information may then be used to produce dental dentures, implants, aligners, crowns, veneers, partials, relines, mouthguards, retainers, and so forth.

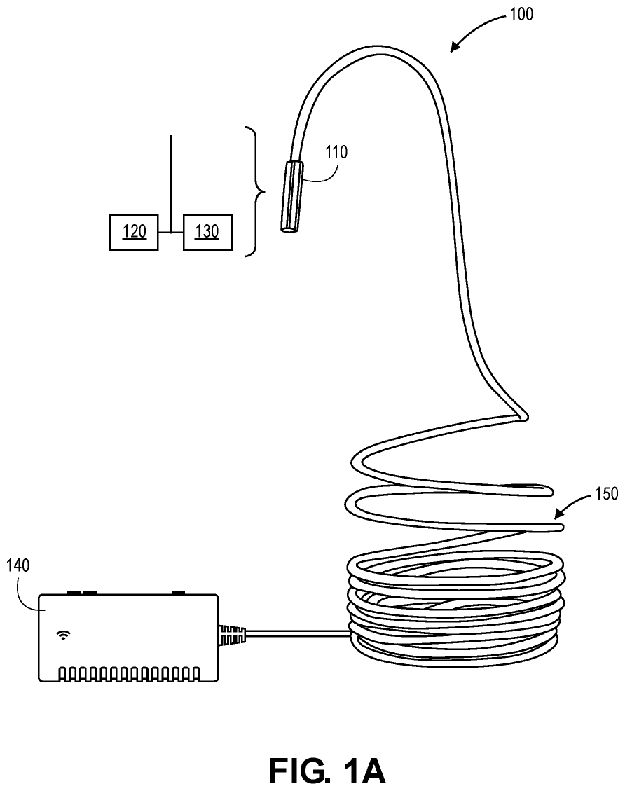

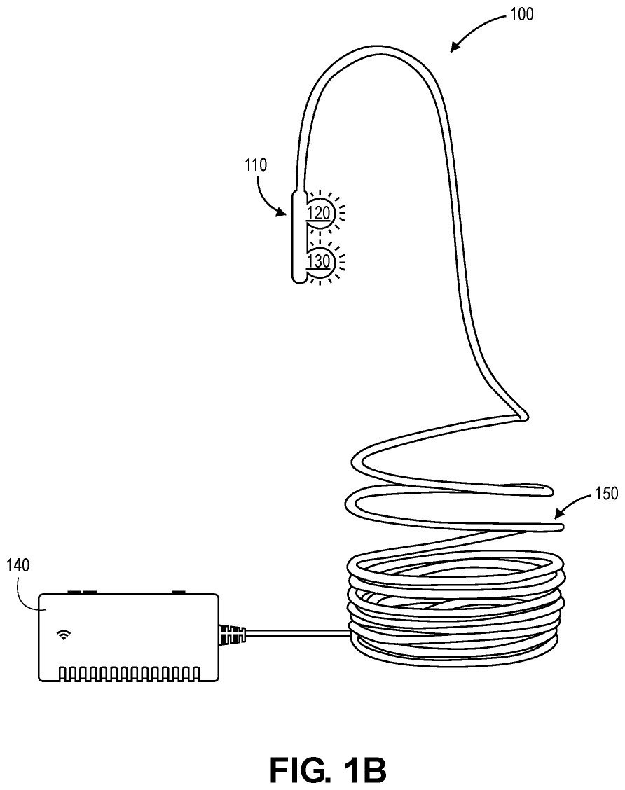

FIG. 1A shows a first example of an intraoral scanning system 100. With reference thereto, system 100 uses a scanner 110 to mobilize the platform using native iOS or android apps running on smartphone 140 (which includes one or more processors, e.g., one or more scanning processors). In some embodiments, the scanner 110 is an infrared scanner 110 including an emitter 120 (also sometimes referred to as a “projector”) and receiver 130 (e.g., a sensor). It is possible to create a 3D model using raw depth feed data (e.g. from an infrared scanner 110 or from a laser scanner 110) instead of an RGB camera. It is also possible to create a 3D model using RGB data and a photogrammetry pipeline. Generally, the raw depth data becomes more accurate as the receiver is moved closer to objects in the mouth, including teeth or gum tissue. Thus, in some examples, a human operator will press the scanner 110 into contact with a patient's teeth or gums. However, holding the scanner 110 a few inches away from a patient's teeth will still produce accurate depth information; and, even if the scanner is a few meters away from the target, depth information may still be obtained. The signal from the receiver 110 can be created by a single paired infrared emitter 120 and receiver 130, which cost less to produce than the RGB camera found on most modern smartphones. For example, an infrared scanner emitter and receiver often cost $4. In some embodiments, the raw depth data is in the form of a point cloud (e.g., a dataset that represents object(s) in space).

Regarding the operation of the scanner 110, the emitter 120 emits dots received by the receiver 130. In some embodiments, the emitter 120 emits around 30,000 dots per second. Further, in some embodiments, a human operator will operate the scanner 110 such that a very large percentage (e.g., 90%) of the dots hit a tooth or other target in a patient's mouth. In this regard, in some embodiments, the smartphone 140 will provide an alarm or indication to the human operator if less than a certain percentage (e.g., 90%) of the dots are not hitting the target.

In addition, although the example of FIG. 1A shows smartphone 140, it should be understood that the data from the scanner 110 may be received and processed by any processing device. In this regard, in the example of FIG. 1A, the signal from scanner 110 is processed by the smartphone 140. This processing produces a raw depth feed, which is an integer, usually measured in microns, indicating the distance between the scanner and the object being scanned (e.g., a tooth or gum). It should be understood that the signal from the scanner 110 may be feed to a printed circuit board (PCB).

FIG. 1B shows an example embodiment with scanner 110 shaped as a toothbrush. This example shows emitter 120 and receiver 130 on the “head” of the “toothbrush.” This example embodiment may make it easier for a human operator to administer a scan to a patient.

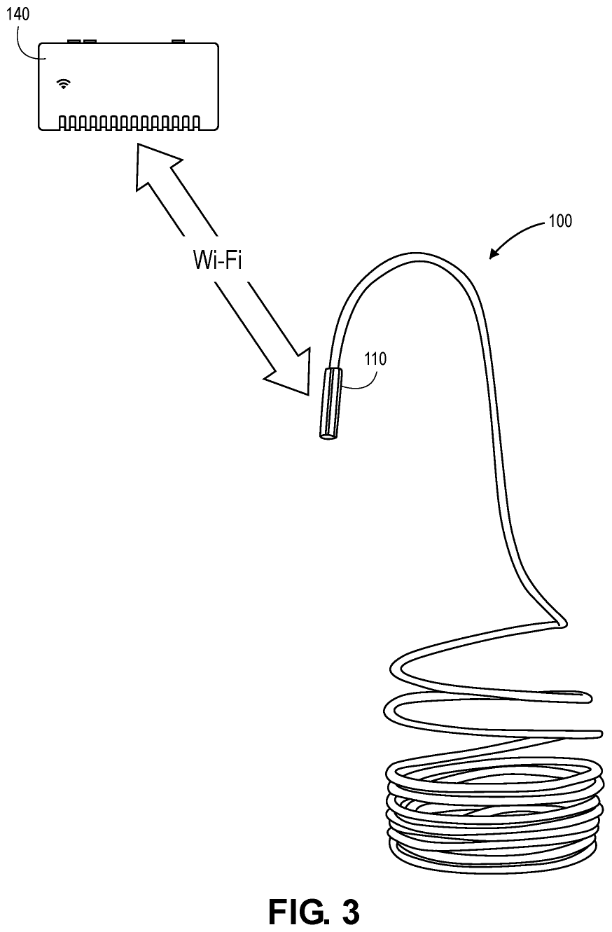

The scanner 110 can connect to the smartphone 140 directly or wirelessly. In direct connection embodiments such as the embodiment of FIG. 1A, the components can be packaged into an easy to maneuver sensor-on-a-stick or snake-like system 150. Wireless connection embodiments require a rechargeable battery or some other source of electrical power, but add to the portability and interoperability of the platform. In this regard, FIG. 3 shows wireless communication between the scanner 110 and the smartphone 140. Any wireless communication method may be used (e.g., wi-fi, Bluetooth etc.).

A native application can connect the raw depth feed or images created by the scanner to an application programming interface (API) that turns the data into a 3D model in the industry standard OBJ and STL formats. The completed scan would be materially similar to those created by incumbent scanning platforms, and dental labs would be unable to detect significant material differences. At best, they include more detail than a scan with only an RGB camera. At worst, they produce workable scans at a small fraction of the cost. If the scanner included an RGB camera it produces colored scans (although this is a purely aesthetic feature). The software may use real-time web communication technologies (such as those used in video chatting applications) to stream data from the smartphone to a server for rendering.

Once scans are created, they can be stored for future retrieval in an S3-compatible object storage system. They will be readily viewable by patients and dentists on mobile and web platforms (e.g., as illustrated in the example of FIG. 5), where they can be used as controls in denture and implant designs.

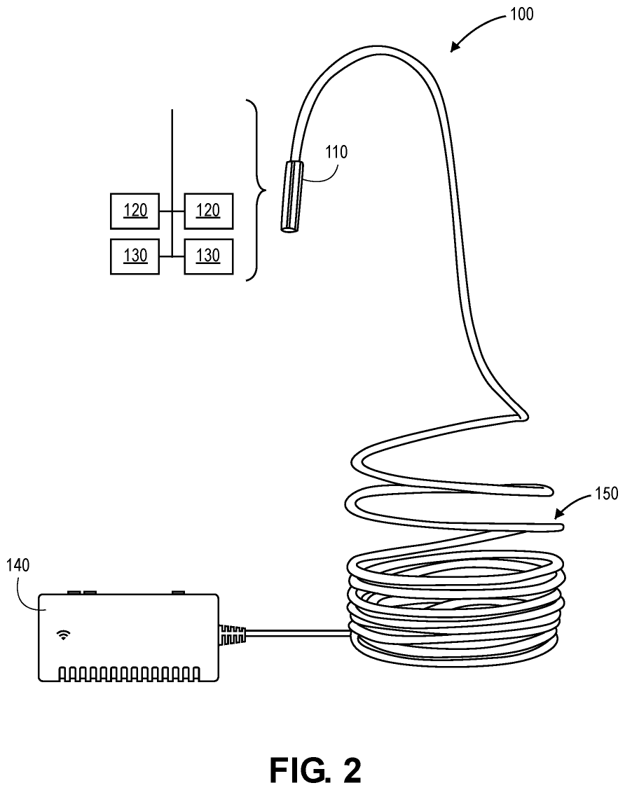

FIG. 2 shows a second example of an intraoral scanning system 200. In the example of FIG. 2, the scanner 110 includes two emitters 120 and two receivers 130. In this regard, although the additional emitter and receiver of this example increase the cost of the system, they decrease the time that a human operator will spend scanning a patient's mouth. For example, the additional emitter and receiver allow for additional data points to be taken while the human operator runs the scanner 110 along a person's teeth. As this shows, further emitters and receivers may be added to reduce the time needed to scan a patient's mouth (although this will increase the cost of the system).

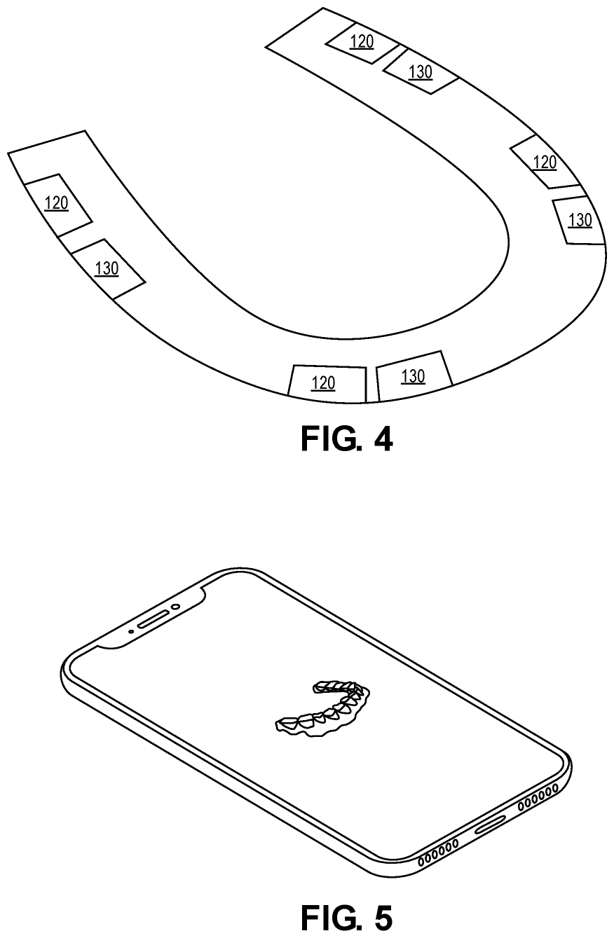

Further regarding additional emitters and receivers, FIG. 4 shows an embodiment involving a mouthguard. In the example of FIG. 4, the mouthguard 400 includes a plurality of emitters 120 and receivers 130. In the example of FIG. 4, four emitters 120 and four receivers 130 are shown; however, the example of FIG. 4 is not limiting and any number of emitters 120 and receivers 130 are possible. As discussed above, adding additional emitters 120 and receivers 130 will decrease the time required to obtain complete scanning information, but will increase the cost.

Further regarding a human operator using the scanner 110, it should be understood that to obtain complete information, the human operator will often run the scanner along the front and back of a patient's upper and lower teeth. In some embodiments, the smartphone 140 may provide real time feedback to the human operator. For example, the smartphone may provide an indication to the human operator that insufficient information has been received regarding a particular tooth or section of the mouth. In practice, for a complete scan of a patient's teeth, a trained human operator will often require two minutes or less of scanning time to acquire sufficient information, whereas a novice operator will often require ten minutes of scanning time to acquire sufficient information.

To further aid the human operator, the scanner 110 may include a replacement head. The replacement head allows for quick sterilization between patients. The replacement head can simply be a shell or other prophylactic that goes over the electric components in the scanner.

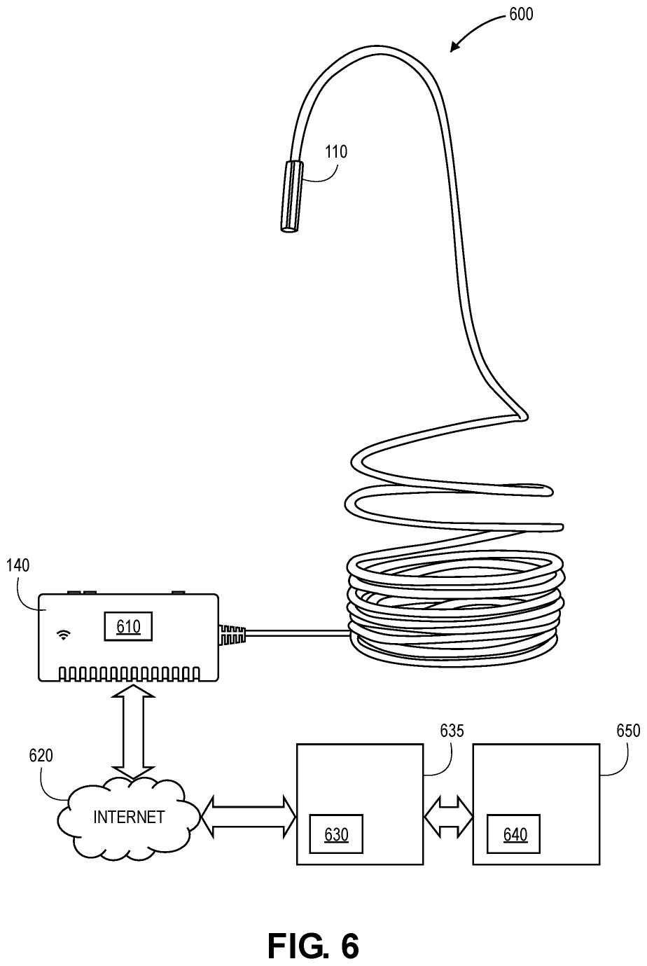

Once sufficient information has been acquired, a dental product may be produced by any method. In some embodiments, the dental product is produced by 3D printing. FIG. 6 shows an example of an intraoral scanning system 600 sending information to a dental lab 635, which in turn sends information to dental manufacturing facility 650. In the example of FIG. 6, the scanning processors 610 of the smartphone 140 receive the scanning signal from the scanner 110. The scanning processors 610 send, through internet 620, the scanning signal to the dental production processors 630. Alternatively, the scanning processors 610 process the scanning signal into raw depth data and sends the raw depth data to the dental manufacturing processors 630. The dental lab 635 produces a design for any dental product, and sends the design to the dental manufacturing facility 650, which controls dental manufacturing equipment 640. In some embodiments, the dental manufacturing equipment 640 is a 3D printer.

It should be understood that the dental lab 635 may be a facility on the cloud. For example, the dental lab 635 may include server(s) which may also include one or more computer memories. The memories may include one or more forms of volatile and/or non-volatile, fixed and/or removable memory, such as read-only memory (ROM), electronic programmable read-only memory (EPROM), random access memory (RAM), erasable electronic programmable read-only memory (EEPROM), and/or other hard drives, flash memory, MicroSD cards, and others. The memories may store an operating system (OS) (e.g., Microsoft Windows, Linux, Unix, etc.) capable of facilitating the functionalities, apps, methods, or other software as discussed herein. The memories may also store machine readable instructions, including any of one or more application(s), one or more software component(s), and/or one or more application programming interfaces (APIs), which may be implemented to facilitate or perform the features, functions, or other disclosure described herein, such as any methods, processes, elements or limitations, as illustrated, depicted, or described for the various flowcharts, illustrations, diagrams, figures, and/or other disclosure herein.

Further regarding the data flow, in some embodiments, the scanner 110 may send the scanning signal directly via wi-fi (or other wired or wireless technique) to a router; and the router may then send, via the internet 620, the data to the dental production processors 630.

In addition, different dental products require depth information of different portions of a patient's mouth. For example, dental aligners may require depth information of a patient's teeth. In another example, a denture may require depth information of only a portion of a patient's gums.

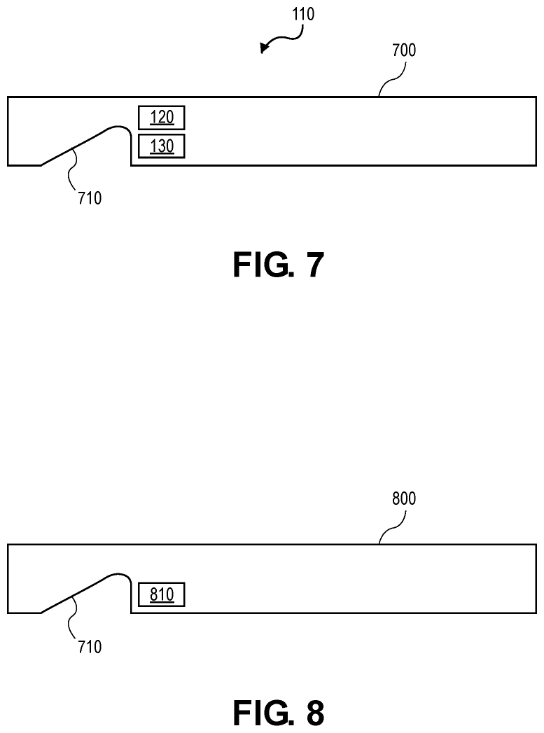

FIG. 7 shows an example embodiment of a handle 700 including a mirror 710, an IR emitter 120, and IR receiver 130. The mirror 710 is at a predetermined (e.g., 45 degree) angle with an axis of the handle 700. In some embodiments, the IR emitter 120, and IR receiver 130 point parallel to the axis of the handle 700, and the emission from the IR emitter (or projector) 120 is reflected 45 degrees by the mirror 710 at the end of the handle 700. It should be understood that the components of the handle 700 can connect to a smartphone, such as the smartphone 140 of FIG. 1, through the techniques discussed above. In some embodiments, the handle 700 is 800 mm long with a 160 mm diameter. In some embodiments, the ends of the handle 700 are flat rather than rounded, which advantageously allows for more room when scanning a patient's mouth, and further advantageously allows for the handle 700 to be propped up on a flat surface such as a table.

FIG. 8 shows an example embodiment of a handle 800 including a mirror 710 and an RGB camera 810. The mirror 710 is at a 45 degree angle with an axis of the handle 800. In some embodiments, the RGB camera 810 requires approximately 3 cm of distance from a target to focus. In some embodiments, the RGB camera 810 points parallel to the axis of the handle 800, and is reflected 45 degrees by the mirror 710 at the end of the handle 700. It should be understood that the components of the handle 800 can connect to a smartphone, such as the smartphone 140 of FIG. 1, through the techniques discussed above. In some embodiments the handle 800 is 800 mm long with a 160 mm diameter. In some embodiments, the ends of the handle 800 are flat rather than rounded, which advantageously allows for more room when scanning a patient's mouth, and further advantageously allows for the handle 800 to be propped up on a flat surface such as a table.

In addition, it should be noted that the scanner 110 of the system and methods disclosed herein may be applied to an endoscopic camera. In some embodiments, the RGB camera 810 uses a macro lens, and in other embodiments a stop lens is used. In some aspects, a macro lens advantageously allows for a better focal distance than a stop lens.

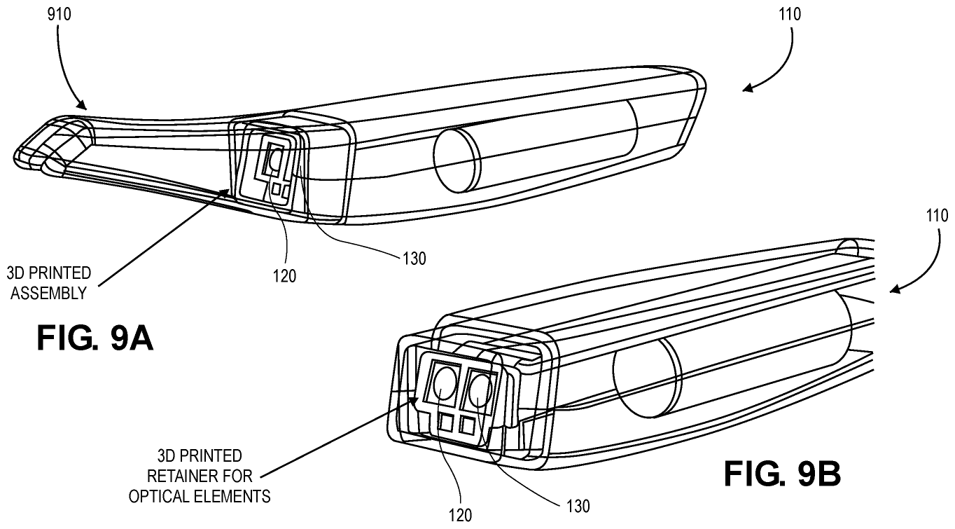

FIG. 9A shows an example of a scanner 110 with a headpiece 910, while FIG. 9B shows an example of a scanner 110 without a headpiece. The scanner 110 includes emitter 120 and receiver 130.

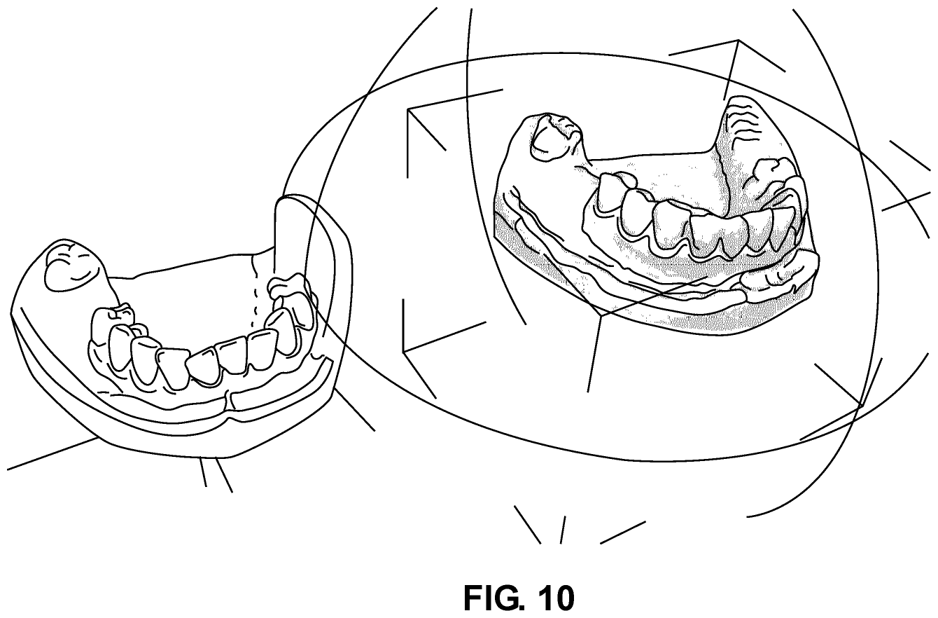

FIG. 10 shows two examples of 3D printed dentures, which may be printed in accordance with any of the techniques described herein.

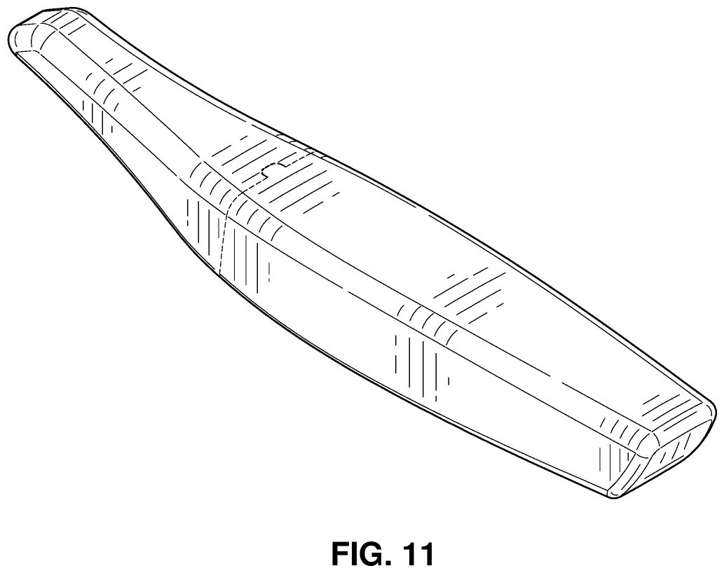

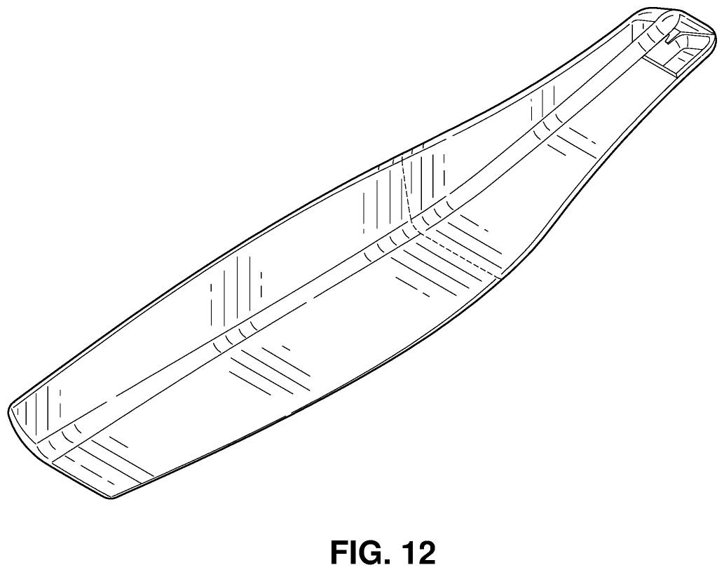

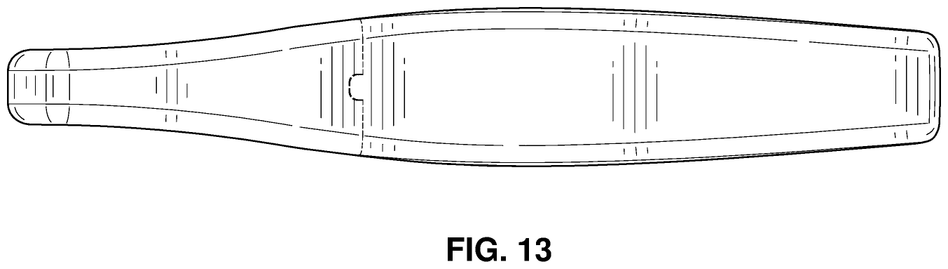

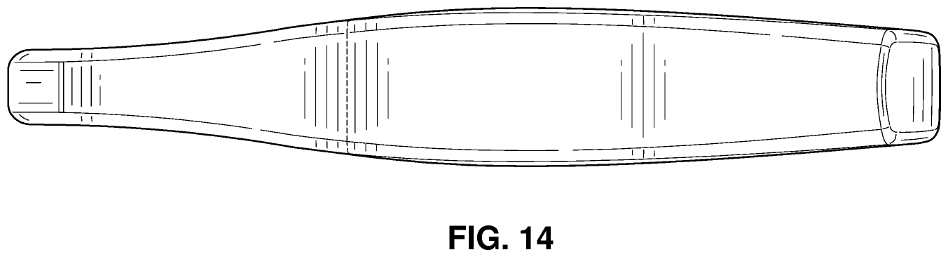

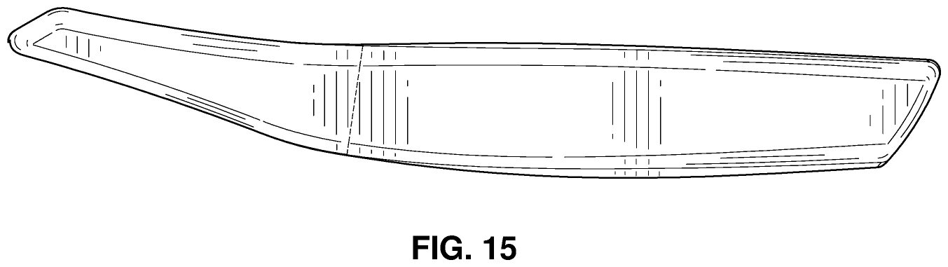

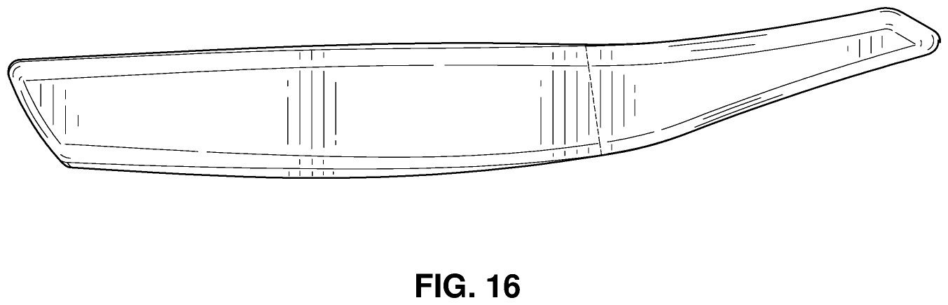

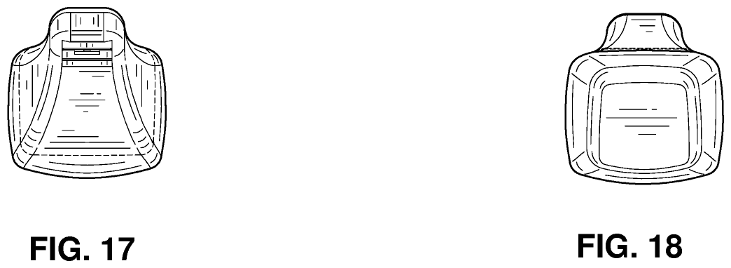

FIGS. 11-18 show additional views of example scanners in accordance with the systems and methods described herein.

### Additional Exemplary Embodiments

Aspect 1. An intraoral scanning system for generating image and/or depth data to be used to produce a dental product, the system comprising:

a scanner communicatively coupled with one or more processors, the scanner comprising: (i) an emitter or projector configured to output an emission, and/or (ii) a receiver configured to receive the emission and generate a scanning signal based on the received emission; and

wherein the one or more processors are configured to generate the depth data based on the scanning signal.

Aspect 2. The intraoral scanning system of aspect 1, wherein the scanner is an infrared and/or RGB scanner, the emitter is an infrared emitter or infrared projector, and the receiver is an infrared or RGB receiver.

Aspect 3. The intraoral scanning system of aspect 1, wherein the scanner is a laser scanner, the emitter is a laser emitter, and the receiver is a laser receiver.

Aspect 4. The intraoral scanning system of any of aspects 1-3, wherein:

the emitter is a first emitter, and the receiver is a first receiver; and

the scanner further comprises (i) a second emitter configured to output a second emission, and (ii) a second receiver configured to receive the second emission and generate a second scanning signal based on the second emission.

Aspect 5. The intraoral scanning system of any of aspects 1-4, wherein the scanner is communicatively coupled to the one or more processors via wi-fi or USB.

Aspect 6. The intraoral scanning system of any of aspects 1-5, further comprising a mouthguard including the scanner, wherein:

the emitter is a first emitter, and the receiver is a first receiver; and

the scanner further comprises (i) a second emitter configured to output a second emission, and (ii) a second receiver configured to receive the second emission and generate a second scanning signal based on the second emission.

Aspect 7. A method for producing a dental product, the method comprising:

emitting, with an emitter of a scanner, an emission;

receiving, with a receiver of the scanner, the emission;

generating a scanning signal based on the received emission;

sending the generated scanning signal to one or more scanning processors;

with the one or more scanning processors, generating depth data of the patient's mouth; and

sending the depth data from the one or more scanning processors to one or more dental production processors.

Aspect 8. The method of aspect 7, further comprising producing the dental product according to the depth data.

Aspect 9. The method of aspect 7, further comprising producing the dental product according to the depth data, and not according to any image data from a camera.

Aspect 10. The method of any of aspects 7-9, wherein the emission is an infrared emission, and the emitting comprises emitting the infrared emission.

Aspect 11. The method of any of aspects 7-9, wherein the emission is a laser emission, and the emitting comprises emitting the laser emission.

Aspect 12. The method any of aspects 7-11, wherein:

the emitter is a first emitter, and the receiver is a first receiver; and

the method further comprises:

emitting, with a second emitter of the scanner, a second emission;

receiving, with a second receiver of the scanner, the second emission;

generating a second scanning signal based on the second emission; and

generating the depth data further based on the depth data.

Aspect 13. The method of any of aspects 7-12, wherein the generated scanning signal is sent to the one or more scanning processors via wi-fi.

Aspect 14. The method of any of aspects 7-13, further comprising producing the dental product:

according to the depth data; and

using a 3D printer.

Aspect 15. The method of any of aspects 7-14, further comprising producing the dental product according to the depth data;

wherein the produced dental product includes one of a dental denture, implant, aligner, crown, veneer, partial, reline, mouthguard, or retainer.

Aspect 16. The method of any of aspects 7-15, wherein the sending the depth data from the one or more scanning processors to the one or more dental production processors comprises:

sending the depth data from the one or more scanning processors to a wi-fi router; and

sending the depth data from the wi-fi router to the one or more dental production processors.

Aspect 17. The method of aspect 7, wherein the depth data comprises a 3D point cloud.

Aspect 18. A method for producing dental products, the method comprising:

emitting, with a first emitter of a first scanner, a first emission;

receiving, with a first receiver of the first scanner, the first emission;

generating a first scanning signal based on the received first emission;

sending the generated first scanning signal to one or more first scanning processors;

with the one or more first scanning processors, generating first depth data of the first patient's mouth;

sending the first depth data from the one or more first scanning processors to one or more dental production processors;

emitting, with a second emitter of a second scanner, a second emission;

receiving, with a second receiver of the second scanner, the second emission;

generating a second scanning signal based on the received second emission;

sending the generated second scanning signal to one or more second scanning processors;

with the one or more second scanning processors, generating second depth data of the second patient's mouth; and

sending the second depth data from the one or more second scanning processors to the one or more dental production processors;

wherein the one or more dental production processors are located remotely from both the first scanning processors and the second scanning processors.

Aspect 19. The method of aspect 18, wherein the one or more dental production processors are part of a cloud facility.

1. An intraoral scanning system for generating image and/or depth data to be used to produce a dental product, the system comprising:
a scanner communicatively coupled with one or more processors, the scanner comprising: (i) an emitter or projector configured to output an emission, and/or (ii) a receiver configured to receive the emission and generate a scanning signal based on the received emission; and
wherein the one or more processors are configured to generate the depth data based on the scanning signal.

2. The intraoral scanning system of claim 1, wherein the scanner is an infrared and/or RGB scanner, the emitter is an infrared emitter or infrared projector, and the receiver is an infrared or RGB receiver.

3. The intraoral scanning system of claim 1, wherein the scanner is a laser scanner, the emitter is a laser emitter, and the receiver is a laser receiver.

4. The intraoral scanning system of claim 1, wherein:
the emitter is a first emitter, and the receiver is a first receiver; and
the scanner further comprises (i) a second emitter configured to output a second emission, and (ii) a second receiver configured to receive the second emission and generate a second scanning signal based on the second emission.

5. The intraoral scanning system of claim 1, wherein the scanner is communicatively coupled to the one or more processors via wi-fi or USB.

6. The intraoral scanning system of claim 1, further comprising a mouthguard including the scanner, wherein:
the emitter is a first emitter, and the receiver is a first receiver; and
the scanner further comprises (i) a second emitter configured to output a second emission, and (ii) a second receiver configured to receive the second emission and generate a second scanning signal based on the second emission.

7. A method for producing a dental product, the method comprising:
emitting, with an emitter of a scanner, an emission;
receiving, with a receiver of the scanner, the emission;
generating a scanning signal based on the received emission;
sending the generated scanning signal to one or more scanning processors;
with the one or more scanning processors, generating depth data of the patient's mouth; and
sending the depth data from the one or more scanning processors to one or more dental production processors.

8. The method of claim 7, further comprising producing the dental product according to the depth data.

9. The method of claim 7, further comprising producing the dental product according to the depth data, and not according to any image data from a camera.

10. The method of claim 7, wherein the emission is an infrared emission, and the emitting comprises emitting the infrared emission.

11. The method of claim 7, wherein the emission is a laser emission, and the emitting comprises emitting the laser emission.

12. The method of claim 7, wherein:
the emitter is a first emitter, and the receiver is a first receiver; and
the method further comprises:
emitting, with a second emitter of the scanner, a second emission;
receiving, with a second receiver of the scanner, the second emission;
generating a second scanning signal based on the second emission; and
generating the depth data further based on the depth data.

13. The method of claim 7, wherein the generated scanning signal is sent to the one or more scanning processors via wi-fi.

14. The method of claim 7, further comprising producing the dental product:
according to the depth data; and
using a 3D printer.

15. The method of claim 7, further comprising producing the dental product according to the depth data;
wherein the produced dental product includes one of a dental denture, implant, aligner, crown, veneer, partial, reline, mouthguard, or retainer.

16. The method of claim 7, wherein the sending the depth data from the one or more scanning processors to the one or more dental production processors comprises:
sending the depth data from the one or more scanning processors to a wi-fi router; and
sending the depth data from the wi-fi router to the one or more dental production processors.

17. The method of claim 7, wherein the depth data comprises a 3D point cloud.

18. A method for producing dental products, the method comprising:
emitting, with a first emitter of a first scanner, a first emission;
receiving, with a first receiver of the first scanner, the first emission;
generating a first scanning signal based on the received first emission;
sending the generated first scanning signal to one or more first scanning processors;
with the one or more first scanning processors, generating first depth data of the first patient's mouth;
sending the first depth data from the one or more first scanning processors to one or more dental production processors;
emitting, with a second emitter of a second scanner, a second emission;
receiving, with a second receiver of the second scanner, the second emission;
generating a second scanning signal based on the received second emission;
sending the generated second scanning signal to one or more second scanning processors;
with the one or more second scanning processors, generating second depth data of the second patient's mouth; and
sending the second depth data from the one or more second scanning processors to the one or more dental production processors;
wherein the one or more dental production processors are located remotely from both the first scanning processors and the second scanning processors.

19. The method of claim 18, wherein the one or more dental production processors are part of a cloud facility.
